(19) United States
(12) Patent Application Publication
(10) Pub. No.: US 2017/0071481 A1
(43) Pub. Date: Mar. 16, 2017

(54) WEARABLE DEVICE FOR PULSE READING

(71) Applicant: BE XIE, Fremont, CA (US)

(72) Inventor: BE XIE, Fremont, CA (US)

(21) Appl. No.: 14/852,538

(22) Filed: Sep. 12, 2015

(51) Int. Cl. A61B 5/0205; A61B 5/00

(52) U.S. Cl. CPC A61B 5/02055; A61B 5/6806; A61B 5/0002; A61B 5/72; A61B 2562/164; A61B 5/02438

(57) ABSTRACT

This invention describes a smart pulse reading device placed on a patient's hand or wrist, with which a pulse reading technique through digitalization and signal processing enables telemedicine such that a patient can receive medical attention. The invention includes the benefit of generating a specific prescription for a patient depending on the medical diagnosis. One of the prescriptions generated, may be traditional Chinese medicine used as an alternate form of treatment. By accurately assessing the patient's pulses, the smart device can determine the type of medical condition the patient is affected by. The device features various sensors that are placed above a critical radial artery in the wrist of a patient, and embedded in the device are the pressure sensors in a matrix orientation. The matrix of sensors can detect the pressure of the patient's pulses by calculating the pressure change over time.

### FIELD OF THE INVENTION

The present invention generally relates to medical diagnostics through telemedicine. More particularly, the present invention relates to traditional Chinese medicine pulse reading techniques through digitalization and signal processing which enables telemedicine diagnostics and prescription generation according to patients' pulse types.

### BACKGROUND OF THE INVENTION

Traditional Chinese Medicine (TCM) comprises a broad range of medicinal practices developed in China and dates back to more than 3,000 years ago. Types of TCM include acupuncture, herbal medicine, massage, and specific types of exercises used in aiding patients with their health conditions. Today, TCM is used as a complementary or alternative treatment along with the standard prescription medicine. The concept of TCM was initially tied to a perceived disharmony in the functions of the human body or the disharmony between the human body and its surrounding environment. This type of therapy was based on identifying the disharmony pattern in the anatomy of the patient. To diagnose a patient, TCM practitioners examined human body parts such as the tongue, the smell of the breath, and strength of pulses on the pulse points of the body to initiate a therapy. Since the discovery of TCM, examination of the human tongue and the pulses of the heart have continued to be the primary sources of diagnostic information for determining health conditions when applying TCM. In general, pulse-reading is considered an art that demands assiduous study and innate talent.

When analyzing the pulse points of the human body, the characteristics of a pulse such as the rhythm, and strength can be directly pointed to specific disease patterns, and thus have been found to be useful in determining the medical treatments.

After a patient is diagnosed, a popular prescription of TCM is herbal tea or soup. In China, there are a vast variety of natural elements such as leaves, seeds, roots, etc., which can be used to treat medical ailments. In many cases, prescribing these varieties of elements can help aid in a patient medical ailment.

In combination with TCM, telemedicine, which refers to the actual delivery of remote clinical services using technology, has now become a more accepted medical practice throughout China and western cultures. With telemedicine being used for medical information exchange from one site to another via electronic communications to improve a patient's clinical health status, combining TCM with telemedicine can further aid in the health benefits for patients requiring remote medical attention.

In TCM, pulse reading is one of the most important techniques used by the medical practitioner. By testing and reading the pulses, the practitioner can collect useful information for medical diagnosis. Together there are a total of 9 places with valuable diagnostic information on each human wrist. There are 28 different qualities of a pulse which a practitioner must be alert for. Different combinations of pulses also have different diagnostic significances. The sheer number of possibilities allows fine distinctions in interpreting the gathered information. In U.S. application Ser. No. 12/378,362 (Publication No. US-2009-0204668), a TCM diagnostic method process is described. The application involves a patient taking the pulse on his/her wrist crease on the radial side of the medial aspect of the forearm over the radial artery. When a pulse is inspected, information is entered into a computer where a pulse data collector sends the pulse readings to a remote server. The remote server then matches the pulse readings to a pre-stored diagnostic pulse reading system. A doctor will receive the pulse readings and determine the type of TCM medication the patient requires based on the pulse reading stored in the remote server. Standardizing the pulse reading using electronic analog and digital devices is helpful for the practitioner or doctor to make a diagnosis by obtaining information collected in the pulse reading.

With advanced communications technologies such as email, smart phones, and other wireless communication tools, today's hospitals, health agencies, and private physicians have continued to adapt the telemedicine technology into their practices. The benefit of telemedicine may be applied to patients who live in remote areas and are at greater distances to primary health care facilities. In addition, without the need to physically attend a doctor's office or heath care center, patients may be able to conveniently communicate through video and email communication to receive medical attention they require.

Other advantages of using telemedicine include the use of live interactive video for communication between patients and physicians to exchange images, display vital signs, and video clips of the patient's symptoms. Furthermore, remote patient monitoring can be utilized with telemedicine as remotely sending data to a home health agency for interpretation. These types of data include specific vital signs such as blood glucose or heart electrocardiogram (ECG) and these services can be used as an alternate to physically attending a health care facility or physician's office in order to receive medical treatment. Telemedicine may also be used as a means for providing continuing education for health professionals and special medical education seminars for targeted groups in remote locations. This type of education, can aid in preventing diseases or sicknesses that may be common to the specific environment where patients live.

In recent years, the continuing development of information technology helped advancing TCM to more areas of medicinal practice. The trends in adopting TCM into mainstream Western medicinal practice has become more common and widely used as well. Although many of the benefits of TCM have not been scientifically proven, the TCM practice has been in the medical community for thousands of years and particularly in China, and continues to be an alternate form of medical practice as it evolves into other areas of the medical field.

As computer technology has advanced with the advent of the internet and broadband networks, concepts such as telehealth and telemedicine have been discovered to aid in further medical advancements. With many different types of telemedicine such as telenursing, telerehabilitation, telepharmacy, and emergency telemedicine to name just a few categories, the opportunity to advance the provision of remote medical attention to patients has become an ever growing field.

Since TCM continues to be a form of medical treatment more commonly used in China and some parts of western culture, it is advantageous to incorporate TCM with telemedicine. In more recent years, the combination of TCM with telemedicine is proving to be an effective method of treating health matters for patients in the Chinese community and other western cultures. The alternate form of medicine such as TCM may be the preferred method of receiving medical attention for many patients. Thus, the option to receive TCM as an alternate to standard medical practice should be available and be able to coexist with traditional western medicine.

What is needed is an advanced telemedicine device which can be used in areas such as the human wrist or fingers to collect pulse readings. An advanced matrix of sensors which can be placed on a human wrist or finger can provide a more precise reading of pulses for a patient. In turn, the doctor can prescribe a more effective prescription based on the pulse readings.

### SUMMARY OF THE INVENTION

The present invention, with respect to the field of telemedicine and TCM, describes an intelligent pulse reading device to be placed on a patient's hand or wrist. With this device, a pulse reading technique through digitalization and signal processing enables telemedicine so that a patient can receive medical attention remotely. Additionally, the invention includes the added benefit of automatically generating a specific prescription for the patient depending on the medical diagnosis. One of the prescriptions generated, may be a TCM which can be used as an alternate form of treating the medical condition. By accurately assessing the patient's pulses, the pulse reading sensor device can aid in determining the type of medical condition the patient is affected by.

The device features various pressure sensors that are placed on a critical radial artery in the wrist of a patient. The pressure sensors in a matrix form can detect the pressure of the patient's pulses by calculating the pressure change over time. The device cancels the possibility of human error when collecting the data reading as previously described in the prior art. By reading the pressure change over time, the pulse reading is calculated by the pulse reading sensor device. The pulse may also be read and processed through pulse waveform changes, and when the information is gathered, the device can calculate a specific type of prescription that is equivalent to the traditional Chinese form of medicine. For the added benefit of accurately diagnosing a patient, auxiliary ports are included in the smart sensor device to read the patient's body temperature and oxygen count in the bloodstream. With the combination of these features, this novel device provides advanced features and accurate readings to prescribe TCM for patients requiring medical attention.

### BRIEF DESCRIPTION OF THE DRAWINGS

FIG. 1 is an illustration of the pulse reading sensor device as it contains two pieces wrapping around the wrist; the pieces can be reused several times on a patient; the sensors on the device are placed above the radial artery of the veins in the wrist.

FIG. 2A is an illustration of one embodiment of the sensor device designed to calculate the pulse readings on a patient's wrist; there is one uniform sensor to cover the majority of the arteries in the wrist including the radial artery of the veins.

FIG. 2B is an illustration of another embodiment of the sensor device designed to calculate the pulse readings on a patient's wrist; in FIG. 2B the sensors are split into three separate rectangles and cover the majority of the arteries in the wrist including the radial artery of the veins.

FIG. 3 is an illustration of the sensor devices from FIG. 2A and FIG. 2B; the sensor devices are turned ninety degrees with respect to the positions of the sensors in FIG. 2A and FIG. 2B.

FIG. 4 is an illustration of the sensor device along with a detailed explanation of the wearable device and some of the main components such as an embedded processor, a battery, a memory, and a wireless input/output interface.

FIG. 5 is an illustration of the components embedded inside the sensor device that allows a smart phone device to interact and communicate with the sensors.

FIG. 6 is an illustration of another preferred embodiment of the invention; the sensors are embedded in a wearable glove; the pulse reading glove has three rectangles of pressure sensors near the fingertips of index finger, middle finger and ring respectively.

### DETAILED DESCRIPTION OF THE INVENTION

While the present invention may be embodied in many different forms, designs or configurations, for the purpose of promoting an understanding of the principles of the invention, reference will be made to the embodiments illustrated in the drawings and specific language will be used to describe the same. It will nevertheless be understood that no limitation or restriction of the scope of the invention is thereby intended. Any alterations and further implementations of the principles of the invention as described herein are contemplated as would normally occur to one skilled in the art to which the invention relates.

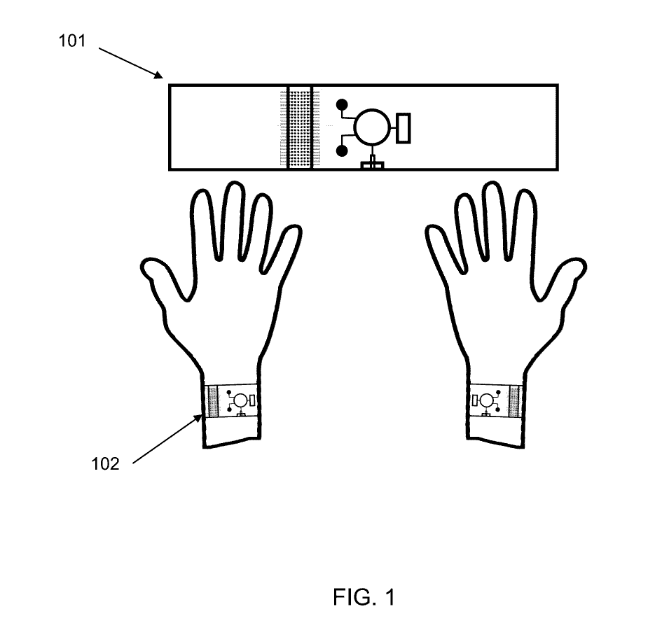

FIG. 1 describes one preferred embodiment as it pertains to the invention. The device is one rectangular sensor device 101 which is made of silicone or soft plastic. The device 101 is extended to show the physical length as compared to an average human wrist 102. In the forthcoming drawings, each of the main components of the device 101 shall be described in further detail, while the overall functionality of the device 101 will also be described.

By viewing the sensor device 101 displayed in its rectangular shape and flat, as an example, the measurement of the length is 8 inches and width is 2 inches. Materials used to manufacture the sensor device 101 can be either plastic or silicone or other materials with flexibility. With silicone or plastic, the shape of the wrist 102 can be accommodated while all the thickness of wrists 102, depending on the patient, an adjustment can be made. Included in the device is a Velcro feature which enables the material to be wrapped around the wrist 102 for full contact between the sensors and the critical arteries of the wrist 102. The entire length of the sensor device 101 can accommodate different wrist thicknesses from various patients. Each sensor device 101 may also be reused for several patients. The device is sturdy and intended for use on several patients. The device 101 can be used by patients in hospitals, health care facilities, and also in their home. Through the advancements in telemedicine, if a patient desires to use the smart sensor device 101 in their own home, the pulse readings can be helpful in diagnosing the patient's medical condition. It is not necessary for a patient to be in a physician's office, hospital, or health care facility to apply the smart sensor device 101. As discussed in the following paragraphs, the smart phone interface of the device 101 allows pulse readings to be transferred to an application on the smart phone which then furthers conveys the pulse readings to a prognosis center. The prognosis center can prescribe the type of Traditional Chinese Medicine as an alternate to the traditional prescription medicine.

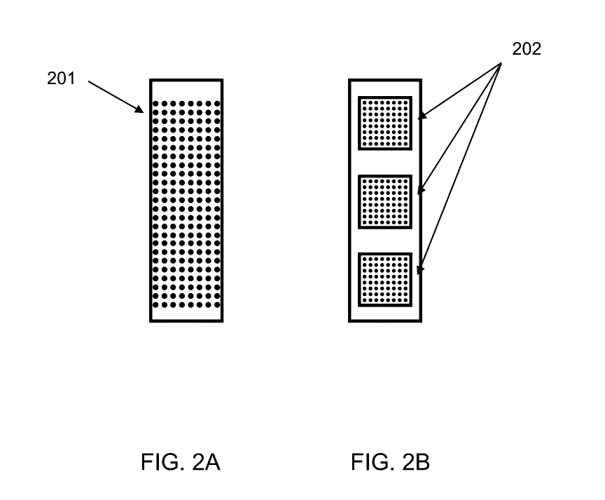

FIG. 2A and FIG. 2B show two configurations of the sensor metrics according to the invention. In FIG. 2A the pressure sensor matrix 201 is designed uniformly across the sensor device. As the sensor device is wrapped around the radial artery of the wrist, the uniform matrix of pressure sensors 201 can be applied to the wrist to touch the skin above the radial artery. When the sensor device is applied around the wrist and activated, the change in pressure of the radial artery over time can be calculated, and the traditional pulse reading measurements are collected. Some of the specific parameters included in the sensor device when reading the pulse include:
Pulse width
Pulse rush or relax
Pulse rate
Pulse depth

In FIG. 2B, the sensor device is designed with the pressure sensors in three separate rectangular pressure sensors 202. In the traditional Chinese culture, for Chinese pulse taking positions, the areas under the three matrixes are called Chun, Guan, and Chi respectively. According to traditional practice, the practitioner uses three fingers to touch and sense these three areas to help diagnose a patient's medical condition. When the pressure sensors 202 are placed over the radial artery of the vein of the wrist, the pulse readings are calculated. The pulse readings are then processed, analyzed and used to aid in diagnosing the medical condition of a patient, and further used to prescribe according to TCM theory.

As described for FIG. 2A, the sensors in FIG. 2B are designed to be positioned directly over the skin above the radial artery. Any of the three sets of sensors can be applied to the artery for collecting pulse readings. The same parameters as applied in FIG. 2A also apply to the orientation of the pressure sensors 202 in FIG. 2B. The pulse width, pulse depth, pulse rate, and pulse rush/relax can be calculated and the readings are collected to determine the type of medical prescription the patient should be prescribed. By using the theory of disharmony pattern from Traditional Chinese Medicine and applying technological advancements in sensor reading devices, the patient can be diagnosed by using the sensor device. Based on the diagnosis a prescription can be provided through the technology of telemedicine.

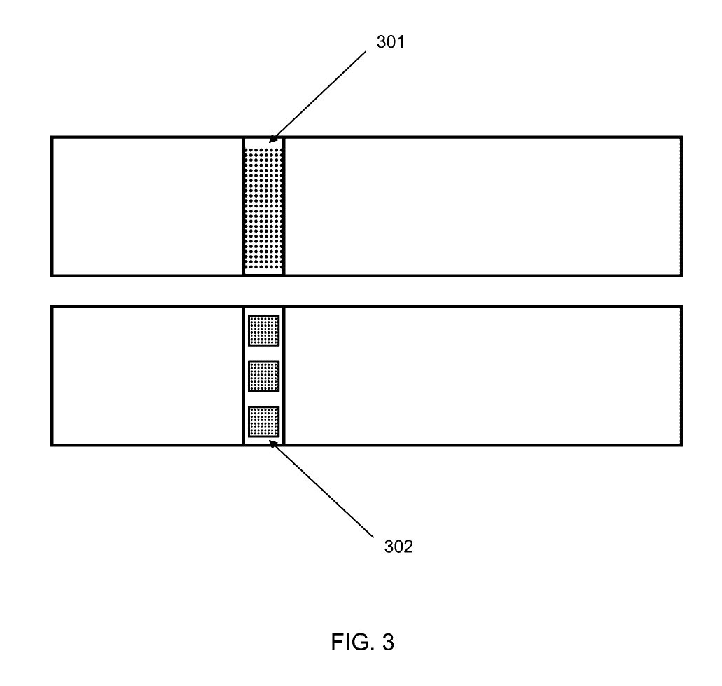

FIG. 3 compares the two matrix styles from FIG. 2A and 2B. In FIG. 3, the orientation of the three rectangles from 202 in FIG. 2A has the enhanced features to benefit the accuracy of the pulse readings.

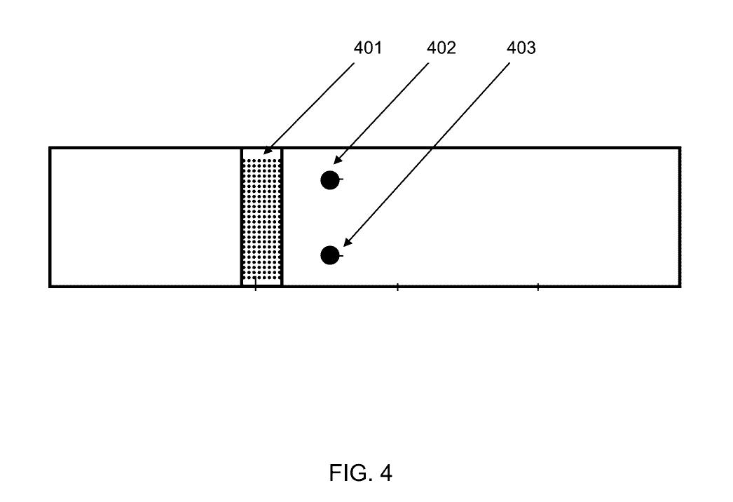

FIG. 4 is an illustration of the sensor device displayed in a flat position. In the rectangular matrix, the pressure sensor 401 is positioned to cover the arterial vein in a patient's wrist. In addition to the pressure sensor 401, there are auxiliary ports on the sensor device to read other medical information which can aid in diagnosing a patient's medical condition. These auxiliary ports includes a body temperature sensor 402 to read the patient's body temperature. Another auxiliary port 402 is the oximeter. With an oximeter sensor, the proportion of oxygenated hemoglobin in the blood can be measured. When combining the oximeter along with the body temperature sensor, a more accurate diagnosis of the patient's medical condition can be determined. When analyzing the human anatomy and human physiology to provide a medical diagnosis, many factors including the oxygen level in the blood, body temperature, and pulse rate can be critical aspects to be considered by nurses, physicians, and health care professionals. With the smart sensor device presented in FIG. 4, and more particularly in the orientation of the pressure sensor 401 matrix, which includes the auxiliary ports to read the body temperature and the oxygen level in the blood, the smart sensor device can serve to accurately diagnose the medical condition for a patient. Once the patient applies the sensor device to the wrist, the pressure sensor 401 is placed over the radial artery of the wrist. The other auxiliary ports are activated to generate a reading for the body temperate along with the oxygen level in the blood stream. The combination of all three readings will aid a nurse, physician, or healthcare expert to more accurately diagnose the patient's medical condition. One of the prescriptions that may be allocated to the patient is a Traditional Chinese Medicine to treat the medical ailment.

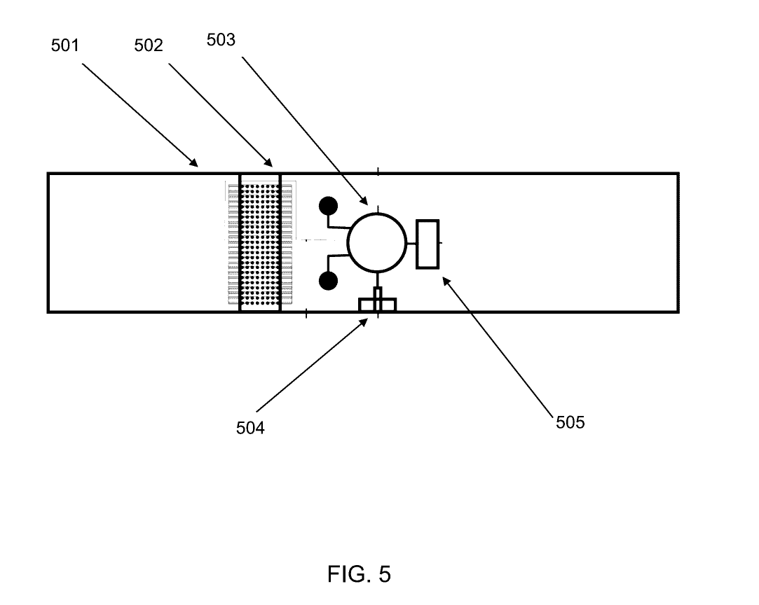

In FIG. 5, the illustration of the sensor device 501 is presented in the same flat position as in previous figures. In this illustration, the pressure sensor 502 matrix is one uniform configuration as opposed to the three rectangle matrix shown in FIG. 2 and FIG. 3. For the purpose of simplicity and describing the components of the invention in detail, FIG. 5 is presented with the sensor device 501 in a flat position. In this position, the pressure sensors 502 are shown in a uniform rectangular matrix. Included in the sensor device 501 are components such as an embedded processor 503, a battery, a wireless input/output interface 504, and a memory chip 505. When a patient wears the smart sensor device 501 around the wrist, the pressure sensor 502 is positioned to touch the skin above the arterial vein of the wrist in order to read the pulses of the patient. As described in FIG. 4, the body temperature auxiliary port along with the oximeter provide two other key readings to aid a nurse, physician, or health care professional in diagnosing a patient's medical condition.

Once information pertaining to the pulses, body temperature, and oxygen level are collected from the smart sensor device 501, the embedded processor 503 is able to process the information and send the results to a smart phone device. This is done through the input/output interface 504 of the smart sensor device 501. A battery is embedded inside the smart sensor device 501 to supply power for operating the embedded processor 503. A memory chip 505 allows the information from the pressure sensor 502, the body temperature port, and the oximeter port to be stored and then transferred the readings to the smart phone application. A smart phone application which can be downloaded onto the smart phone can be activated to process the information and provide the results of the pulses, body temperature, and blood oxygen levels. Since processing the pulses of the patient is one preferred method of diagnosing a patient with Traditional Chinese Medicine principles, the smart phone application provides the pulse information necessary in order to determine the TCM prescription.

Once the patient's pulse readings are processed, another factor to be considered is the patient's verbal explanation of the medical condition. The pulse reading and verbal communication of the patient for the medical condition combine into the two intermediate steps before final diagnosis of the patient's medical condition. A disease diagnosis application from the smart phone app takes the collected information including the pulse reading and patient's verbal communication of the medical condition to provide the final diagnosis. Based on the final medical diagnosis, a Traditional Chinese Medicine prescription can be suggested to the patient as an alternate form of medication. A Chinese herb formula for example can be prescribed along with the traditional prescription medicine. In the Chinese culture, the varieties of tea produced from the evergreens are believed by the Chinese to be useful in aiding with medical conditions. The types of tea which include, oolong, white, and green tea have been shown to carry many health benefits for humans. Green tea, for example has been shown to improve blood flow and lower cholesterol. Recent studies have found that green tea helped preventing a range of heart-related health issues including high blood pressure to congestive heart failure. With the use of the smart sensor device in the present invention, a natural herbal green tea can be prescribed to assist in healing a patient with a medical condition. The smart sensor device of the present invention gives the patient confidence that the prescription is accurate based on the symptoms and medical readings from the sensor device. Thus, a Chinese herbal tea such as the green tea can be prescribed as an all natural alternative to traditional prescriptions.

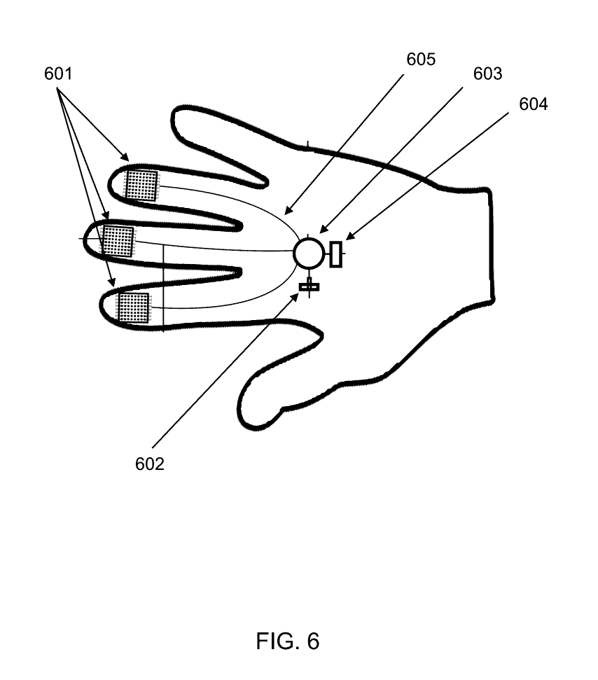

In FIG. 6, another preferred embodiment of the present invention is shown. In this figure, the smart sensor device is worn over the hand similar to a glove. The glove gently covers the hand, and is stretchable to fit around various sizes of patients' hands. With the pulse reading glove, there are three pressure sensors 601 in the shape of rectangles. These pressure sensors 601 operate in the same way as the pressure sensors in the previous figures. They are pressure sensors 601 for reading the pulse of a patient. In FIG. 6, the difference between the wrist wrap sensor device and the glove is that these rectangles are positioned near the fingertips of the patient's index finger, middle finger, and ring finger, respectively. Each pressure sensor 601 is in a rectangular shape, similar to the rectangular matrix pressure sensors described in FIG. 2. With the glove's pressure sensors 601 designed to read the pulse of the patient, the glove can be placed on either the left hand or the right hand. If the patient chooses to wear the glove on the right hand, the rectangular pressure sensors 601 are placed on the right index, middle, and ring finger. Once the glove is worn on the right hand, the patient shall place the three fingers that have the pressure sensors 601 on the left wrist radial artery. In this manner, the left hand pulse can be read through the rectangular pressure sensors 601 on the right hand glove.

Other key components in the glove similar to the wrist wrap pressure sensor, is the input/output interface 602, the processor 603, and the memory chip 604. When the glove is worn on a patient's right hand, the three fingers which align with the three pressure sensors 601 are placed on the patient's left hand. Once the pulse readings are processed, the results are sent to the processor 603 via an electronic wire 605. The processor 603 can also send the information through to the input/output interface 602. Information can also be stored in the memory chip 604 and sent to the input/output interface 602 as well. When the smart phone application is activated, the smart phone can be attached to the glove's input/output interface 602.

The results of the patient's readings are then displayed on the smart phone application. The combination of the patient's verbal communication of the medical ailment, along with the pulse reading from the smart sensor glove allows the patient's medical condition to be diagnosed. Information from the smart phone application can be sent by the patient, nurse, physician, or healthcare expert using telemedicine to a prognosis center. From there, the information gathered which is helpful to prescribe a Traditional Chinese Medicine, can be provided. As described above, a more common TCM that can be prescribed to a patient is the herbal Chinese tea medicine. For instance, when the pulse readings are studied, the TCM prescribed can be a specific type of tea. If the patient is suffering from high cholesterol, the benefits of drinking green tea can be helpful in lowering the patient's cholesterol level. Another example can be fatigue or muscle pain from certain causes. When the patient is suffering from this type of medical condition, the readings from the smart glove sensing device or wrist wrap sensing device can be useful for prescribing a specific type of massaging therapy to aid the patient.

Although one or more embodiments of the smart sensing device have been described in detail, one of ordinary skill in the art will appreciate the modifications to the material selection, design of the smart sensing device for telemedicine prescriptions, particularly for Traditional Chinese Medicine prescriptions. It is acknowledged that obvious modifications will ensue to a person skilled in the art. The claims which follow will set out the full scope of the invention.

1. A device disposed on a human body for measuring physiological parameters, comprising:
a flexible material to be secured on the human body and covering a portion of the human body;
a sensor set disposed on the flexible material and in contact with the human body, wherein the sensor set measures at least one of physiological parameters including a arterial pulsation and generate a plurality of electrical signals representing the physiological parameter.

2. The device of claim 1, wherein the sensor set includes a primary sensor and a secondary sensor for measuring different physiological parameters, the primary sensor and the secondary sensor are disposed on different portions of the flexible material and in contact with different portions of the human body.

3. The device of claim 2, wherein the primary sensor measures arterial pulsations, the secondary sensor measures at least one of the physiological parameters including a body temperature and an oxygen saturation level.

4. The device of claim 1, wherein the sensor set includes at least a first sensor subset and a second sensor subset for measuring the arterial pulsation, the first sensor subset and the second sensor subset are disposed on different portions of the flexible material to be in contact with different portions of the human body.

5. The device of claim 4, wherein the flexible material is into a glove to be worn by a user, the first sensor is disposed on a first finger portion of the glove and the second sensor is disposed on a second finger portion of the glove.

6. The device of claim 1, further comprising:
a microprocessor connected to the sensor set, wherein the microprocessor receives the electrical signals and generates a measurement file; and
a memory device connected to the microprocessor, wherein the memory device receives and store the measurement file.

7. The device of claim 6, further comprising a control interface connected to the microprocessor, wherein a user can control an operation of the microprocessor using the control interface.

8. The device of claim 6, further comprising a communication device connected to the microprocessor and the memory device, wherein the communication device receives the measurement file and generates a plurality of communication signals based on the measurement file.

9. A method for measuring physiological parameters from a human body, comprising:
securing a flexible material on the human body, wherein the flexible material covers a portion of the human body;
disposing a sensor set on the flexible material to make contact with the human body;
measuring at least one of physiological parameters of the human body using the sensor set, wherein the physiological parameters include arterial pulsations; and
generating a plurality of electrical signals representing the physiological parameter measured with the sensor set.

10. The method of claim 9, wherein the sensor set includes a primary sensor and a secondary sensor, the step of disposing the sensor set includes:
disposing the primary sensor and the second sensor on different portions of the flexible material;
arranging the primary sensor and the second sensor to make contacts with different portions of the human body; and
taking measurements of different physiological parameters of the human body.

11. The method of claim 10, wherein the step of taking measurements includes:
measuring arterial pulsations with the primary sensor; and
measuring at least one of the physiological parameters including a body temperature and an oxygen saturation level with the secondary sensor.

12. The method of claim 9, wherein the sensor set includes a first sensor subset and a second sensor subset for measuring the arterial pulsation, the step of disposing the sensor set includes disposing the first sensor subset and the second sensor subset on different portions of the flexible material to make contacts with different portions of the human body.

13. The method of claim 12, wherein the step of disposing the sensor set includes:
making a glove using the flexible material;
disposing the first sensor on a first finger portion of the glove; and
disposing the second sensor on a second finger portion of the glove.

14. The method of claim 9, further comprising:
connecting a microprocessor to the sensor set to receive the electrical signals and generate a measurement file; and
connecting a memory device to the microprocessor, wherein the memory device receives and stores the measurement file.

15. The method of claim 14, further comprising:
connecting a control interface to the microprocessor; and
controlling an operation of the microprocessor using the control interface.

16. The method of claim 14, further comprising:
connecting a communication device to the microprocessor and the memory device to receive the measurement file; and
generating a plurality of communication signals based on the measurement file using the communication device.

17. A system for making medical diagnosis, comprising:
a device disposed on a human body for measuring physiological parameters, comprising:
a flexible material to be secured on the human body and covering a portion of the human body;
a sensor set disposed on the flexible material and in contact with the human body, wherein the sensor set measures at least one of physiological parameters including a arterial pulsation and generate a plurality of electrical signals representing the physiological parameter;
a microprocessor connected to the sensor set, wherein the microprocessor receives the electrical signals and generates a measurement file; and
communication device connected to the microprocessor, wherein the communication device receives the measurement file and generates a plurality of communication signals based on the measurement file; and
a remote station for receiving the communication signals, wherein the remote station includes:
a remote memory device having a plurality of standardized measurement files and a plurality of treatment files; and
a remote processor connected to the remote memory device, wherein the remote processor compares the communication signals and the standardized measurement files, the remote processor then selects at least one treatment file based on the comparison result.

18. The communication system of claim 17, wherein the remote station includes a handheld communication device.

19. The communication system of claim 17, wherein the sensor set includes a primary sensor and a secondary sensor for measuring different physiological parameters, the primary sensor and the secondary sensor are disposed on different portions of the flexible material to make contacts with different portions of the human body, the primary sensor measures arterial pulsations, the secondary sensor measures at least one of the physiological parameter including a body temperature and an oxygen saturation level.

20. The communication system of claim of claim 17, wherein the sensor set includes at least a first sensor subset and a second sensor subset for measuring the arterial pulsation, the first sensor subset and the second sensor subset are disposed on different portions of the flexible material to make contacts with different portions of the human body, the flexible material is into a glove to be worn by a user, the first sensor is disposed on a first finger portion of the glove and the second sensor is disposed on a second finger portion of the glove.
